(19) United States
(12) Patent Application Publication
(10) Pub. No.: US 2021/0060099 A1
(43) Pub. Date: Mar. 4, 2021

(54) NUTRITIONAL SUPPLEMENTS AND METHODS OF NUTRITIONAL SUPPLEMENTATION AFFECTING MICROBIOME METABOLISM

(71) Applicant: Amare Global, Irvine, CA (US)

(72) Inventor: Shawn M. TALBOTT, Draper, UT (US)

(73) Assignee: Amare Global, Irvine, CA (US)

(21) Appl. No.: 17/013,424

(22) Filed: Sep. 4, 2020

Related U.S. Application Data
(60) Provisional application No. 62/895,924, filed on Sep. 4, 2019.

(51) Int. Cl. A61K 35/747; A61K 35/745; A61K 31/702; A61K 31/736; A61K 36/73; A61K 36/87; A61K 36/15; A61P 1/00

(52) U.S. Cl. CPC A61K 35/747; A61K 35/745; A61K 31/702; A61P 1/00; A61K 36/73; A61K 36/87; A61K 36/15; A61K 31/736

(57) ABSTRACT

Nutritional supplements and methods of nutritional supplementation are described for improving microbiome balance in a subject. The methods include administering to a subject an effective amount of a composition including a probiotic including at least one species of probiotic bacterium, a prebiotic including at mixture of at least one of isomaltooligosaccharide, galactooligosaccharide and/or galactomannan, and a phytonutrient including a mixture of at least one of apple fruit extract, grapeseed extract, and/or pine bark extract.

### BACKGROUND

The following relates generally to nutritional supplements and to methods of nutritional supplementation of subjects. More specifically, the following relates to nutritional supplements affecting microbiome balance and to methods of supplementation off subjects in need thereof.

In recent years, research has compiled increasing evidence that the gut microbiome of a subject influences the communication between that subject's central and enteric nervous systems. This communication appears to travel both directions, and an imbalance of a subject's gut flora, has been associated with a variety of complaints, ranging from minor discomforts to significant CNS and gastrointestinal disorders.

Treatments for bacterial imbalances traditionally employ antiobiotic medications including ciprofloxacin, rifaximin and/or co-trimoxazole, among other clinical interventions. Although these medications may be beneficial in the near term, they may be poorly tolerated by patients, and may also be unsuitable for long term use.

There is a need for nutritional supplements and methods of nutritional supplementation to treat, improve, cure and/or prevent microbiome balance in patients in need thereof.

### SUMMARY

This application describes improved nutritional supplements and methods of nutritional supplementation that affect microbiome metabolism and/or microbiome balance. Generally, the described techniques provide for unique nutritional supplements and methods of their use to improve microbiome balance in a subject, and to positively influence that subject's health, metabolism and mood.

In some aspects, a method for improving microbiome balance in a subject is described. The method may include administering to the subject an effective amount of a composition including a probiotic including at least one species of probiotic bacterium, a prebiotic including at mixture of at least one of isomaltooligosaccharide, galactooligosaccharide and/or galactomannan, and a phytonutrient including a mixture of at least one of apple fruit extract, grapeseed extract, and/or pine bark extract.

In some instances, the probiotic comprises at least one of Lactobacillus rhamnosus R0011, Bifidobacterium longum R0175, and Lactobacillus helveticus R0052.

In some instances, the probiotic comprises a mixture of at least two of Lactobacillus rhamnosus R0011, Bifidobacterium longum R0175, and Lactobacillus helveticus R0052.

In some instances, the prebiotic comprises at least two of isomaltooligosaccharide, galactooligosaccharide and/or galactomannan In some, the prebiotic comprises at least about 5 grams of at least two of isomaltooligosaccharide, galactooligosaccharide and/or galactomannan.

In some methods, the phytonutrient comprises at least two of apple fruit extract, grapeseed extract, and/or pine bark extract.

Some examples of the methods further include the step of identifying a subject in need of treatment. In some instances, this includes identifying a subject in need of at least one of body fat reduction, blood cholesterol reduction, blood triglyceride reduction, cortisol reduction, mood improvement and/or increases in beneficial gut bacterial species.

Methods for improving microbiome balance in a subject are described. The methods may include administering to the subject an effective amount of a composition including a probiotic mixture including at least two of Lactobacillus rhamnosus R0011, Bifidobacterium longum R0175, and Lactobacillus helveticus R0052, a prebiotic including at mixture of at least one of isomaltooligosaccharide, galactooligosaccharide and/or galactomannan, and a phytonutrient including a mixture of at least one of apple fruit extract, grapeseed extract, and/or pine bark extract.

In some examples of the method and nutritional supplement described herein, identifying a subject in need of treatment includes identifying a subject in need of at least one of body fat reduction, blood cholesterol reduction, blood triglyceride reduction, cortisol reduction, mood improvement and/or increases in beneficial gut bacterial species.

A method for improving microbiome balance in a subject is described. The method may include administering to the subject an effective amount of a composition including, a probiotic including at least about 3 billion CFU of a mixture of Lactobacillus rhamnosus R0011, Bifidobacterium longum R0175, and Lactobacillus helveticus R0052, a prebiotic including at mixture of isomaltooligosaccharide, galactooligosaccharide and/or galactomannan, and a phytonutrient including a mixture of apple fruit extract, grapeseed extract, and/or pine bark extract.

In other aspects, nutritional supplements are described. Such supplements may include a probiotic comprising at least one species of probiotic bacterium, a prebiotic comprising at mixture of at least one of isomaltooligosaccharide, galactooligosaccharide and/or galactomannan, and a phytonutrient comprising a mixture of at least one of apple fruit extract, grapeseed extract, and/or pine bark extract.

In some aspects, the probiotic comprises at least one of Lactobacillus rhamnosus, Bifidobacterium longum, and Lactobacillus helveticus. In some aspects, the Lactobacillus rhamnosus is Lactobacillus rhamnosus R0011. In some aspects, the Bifidobacterium longum is Bifidobacterium longum R0175. In some aspects, the Lactobacillus helveticus is Lactobacillus helveticus R0052.

In some aspects, the probiotic comprises a mixture of at least two of Lactobacillus rhamnosus R0011, Bifidobacterium longum R0175, and Lactobacillus helveticus R0052.

In some aspects, the prebiotic comprises at least two of isomaltooligosaccharide, galactooligosaccharide and/or galactomannan In some aspects, the prebiotic comprises at least about 5 grams of at least two of isomaltooligosaccharide, galactooligosaccharide and/or galactomannan.

In some aspects, the phytonutrient comprises at least two of apple fruit extract, grapeseed extract, and/or pine bark extract.

In some aspects, the nutritional supplement includes a probiotic comprising at least one of Lactobacillus rhamnosus, Bifidobacterium longum, and Lactobacillus helveticus, a prebiotic comprising at mixture of at least one of isomaltooligosaccharide, galactooligosaccharide and/or galactomannan, and a phytonutrient comprising a mixture of at least one of apple fruit extract, grapeseed extract, and/or pine bark extract.

In some aspects, the Lactobacillus rhamnosus is Lactobacillus rhamnosus R0011.

In some aspects, the Bifidobacterium longum is Bifidobacterium longum R0175.

In some aspects, the Lactobacillus helveticus is Lactobacillus helveticus R0052.

In some aspects, the probiotic comprises a mixture of at least two of Lactobacillus rhamnosus R0011, Bifidobacterium longum R0175, and Lactobacillus helveticus R0052.

In some aspects, the prebiotic comprises at least two of isomaltooligosaccharide, galactooligosaccharide and/or galactomannan. In others, the prebiotic comprises at least about 5 grams of at least two of isomaltooligosaccharide, galactooligosaccharide and/or galactomannan.

In some aspects, the phytonutrient comprises at least two of apple fruit extract, grapeseed extract, and/or pine bark extract.

In some instances, the nutritional supplement includes a probiotic comprising a mixture of Lactobacillus rhamnosus R0011, Bifidobacterium longum R0175, and Lactobacillus helveticus R0052, a prebiotic comprising at mixture of isomaltooligosaccharide, galactooligosaccharide and/or galactomannan, and a phytonutrient comprising a mixture of apple fruit extract, grapeseed extract, and/or pine bark extract.

### BRIEF DESCRIPTION OF THE DRAWINGS

FIG. 1 is a table showing the effect on microbiome composite score and individual microbiome species pre- and post-supplementation with the supplement compositions of the present disclosure.

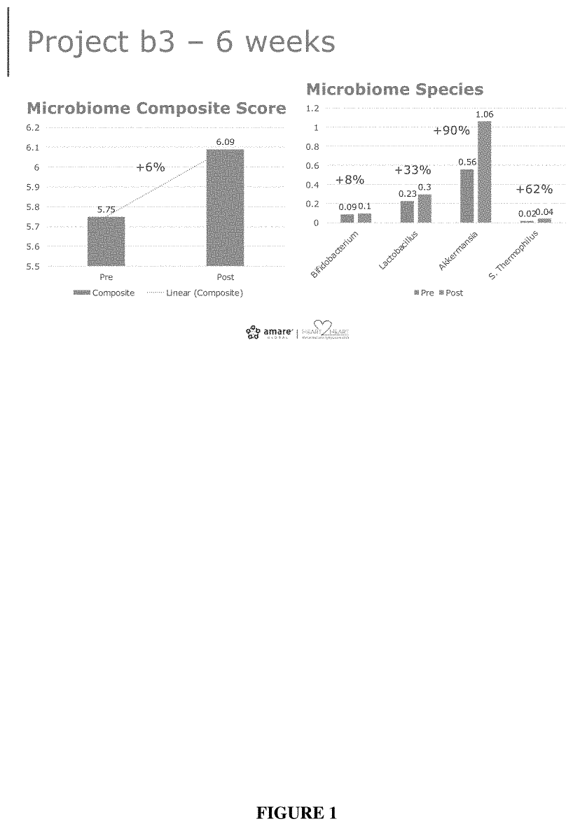

FIG. 2 is a table showing effects on blood chemistry and cardiac risk pre- and post-supplementation with the supplement compositions of the present disclosure.

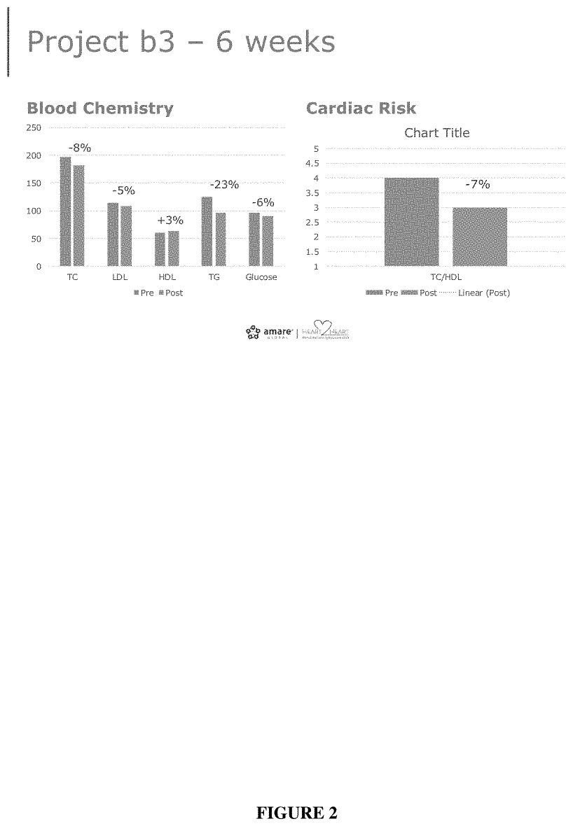

FIG. 3 is a table showing effects on butyrate kinase levels and cortisol levels pre- and post-supplementation with the supplement compositions of the present disclosure.

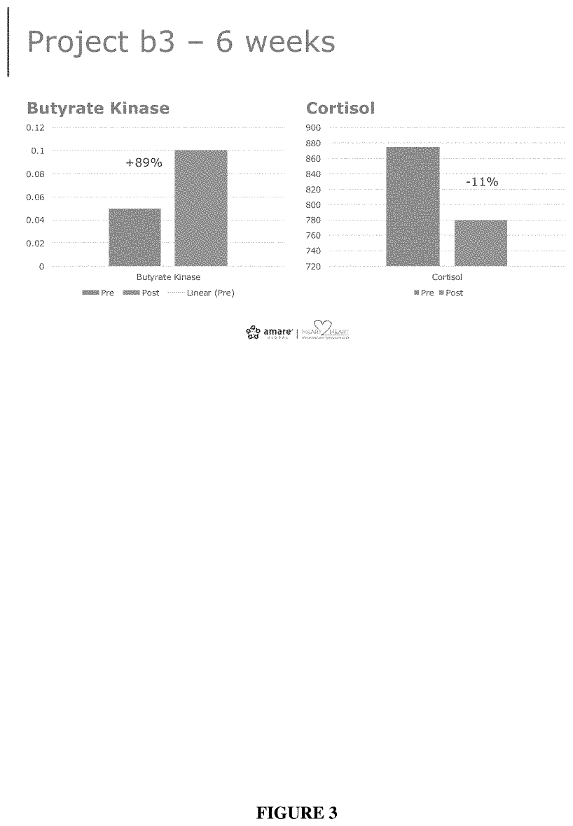

FIG. 4 is a table showing effects on body weight, muscle mass and body fat percentage pre- and post-supplementation with the supplement compositions of the present disclosure.;

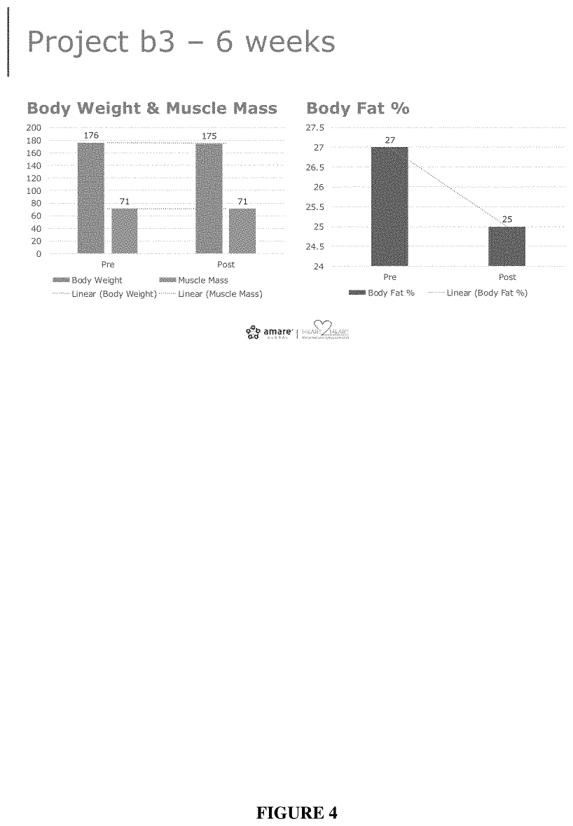

FIG. 5 is a table showing the effect on metabolic ratio and F/B ratio pre- and post-supplementation with the supplement compositions of the present disclosure.

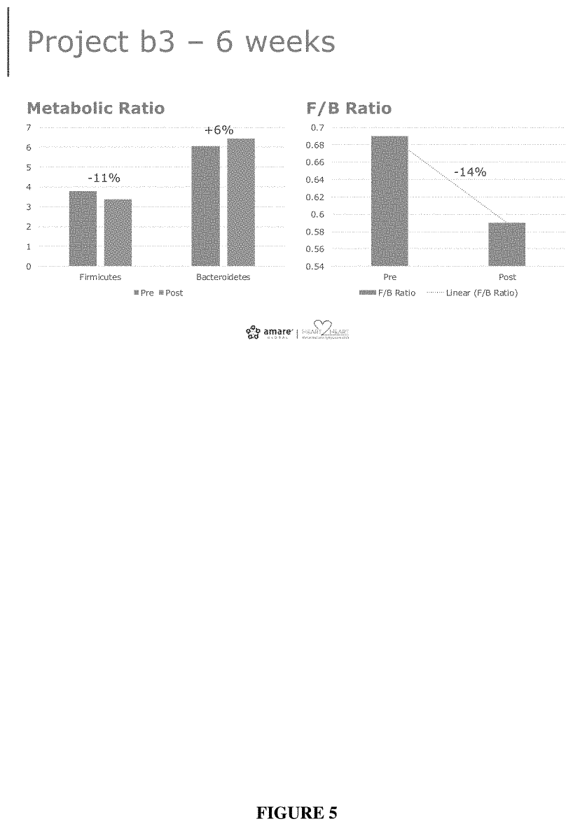

FIG. 6 is a table showing the effect on POMS subclasses and global mood state pre- and post-supplementation with the supplement compositions of the present disclosure.

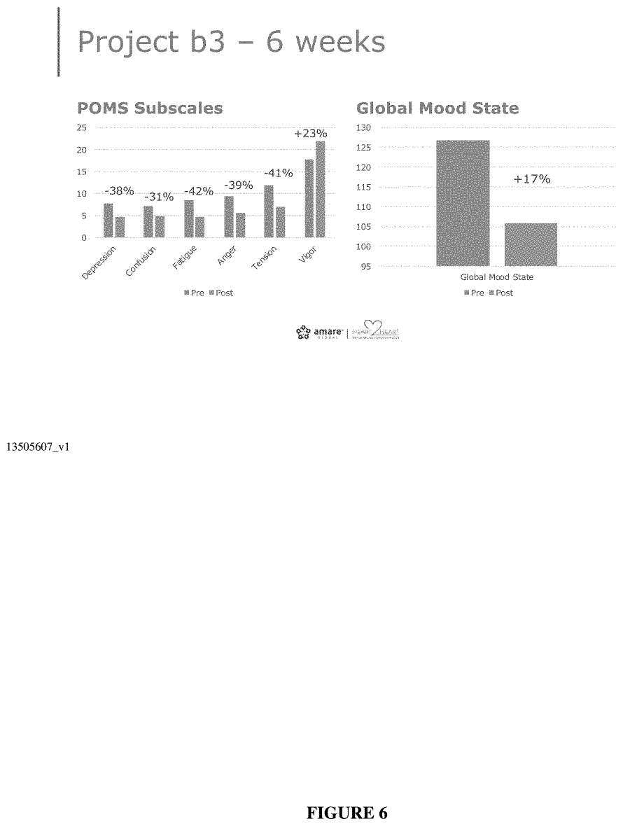

### DETAILED DESCRIPTION

This application describes compositions useful as improved nutritional supplements and methods of their use in supplementing the nutrition of patients to change microbiome balance and to provide positive changes in their health, metabolism, and mood.

Amounts, concentrations, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, an amount of from 1 mg to 200 mg should be interpreted to include not only the explicitly recited limits of 1 mg and about 200 mg, but also to include individual amounts such as 2 mg, 3 mg, 4 mg, and sub-ranges such as 10 mg to 50 mg, 20 mg to 100 mg, etc. Unless otherwise stated, all ranges include both endpoints.

The terms “negative mood state” and “positive mood state” are defined as described using the Profile of Mood States (“POMS”) scale.

As used herein, treatment means any manner in which the symptoms or pathology of a condition, disorder or disease are ameliorated or otherwise beneficially altered. Treatment also encompasses any pharmaceutical use of the compositions herein.

The supplement compositions of the present disclosure may be administered in a variety of suitable dosage forms, including, without limitation, tablets, capsules, granules, powders, liquids, liposome inclusions, ointments, gels, external powders, sprays, inhalable powders, injectable preparations (solutions, suspensions, emulsions, solids to be dissolved when used, etc.), eye drops, eye ointments, suppositories, and the like can be selected appropriately depending on the administration method, and the compositions of the present disclosure can be accordingly formulated. Formulation in general is described in references including Comprehensive Medicinal Chemistry, Volume 5, Editor Hansch et al, Pergamon Press 1990.

As used herein, any range set forth is inclusive of the end points of the range unless otherwise stated.

As used herein, “effective amount” refers to an amount of a substance which is sufficient to achieve its intended purpose or effect. Various biological factors may affect the ability of a delivered substance to perform its intended task. Therefore, an “effective amount” may be dependent on such biological factors. An effective amount of a compound for treating a disorder is an amount that is sufficient to ameliorate, or in some manner reduce a symptom or stop or reverse progression of a condition. Such amount may be administered as a single dosage or may be administered according to a regimen whereby it is effective. The achievement of therapeutic effects may be measured by a physician or other qualified medical personnel using evaluations known in the art, (for example with testosterone supplementation therapy, physical examination, blood and saliva tests may be used), it is recognized that individual variation and response to treatments may make the achievement of therapeutic effects a subjective decision.

As used herein, “administration,” and “administering” may be used interchangeably, and refer to the act of presenting, applying, or introducing a drug to a subject in order to achieve a desired physiological or psychological response.

As used herein, amelioration of the symptoms of a particular disorder by administration of a particular pharmaceutical composition refers to any lessening, whether permanent or temporary, lasting or transient that can be attributed to or associated with administration of the composition.

In some aspects, a method for improving microbiome balance in a subject is described. The method may include administering to the subject an effective amount of a composition including a probiotic including at least one species of probiotic bacterium, a prebiotic including at mixture of at least one of isomaltooligosaccharide, galactooligosaccharide and/or galactomannan, and a phytonutrient including a mixture of at least one of apple fruit extract, grapeseed extract, and/or pine bark extract.

In some instances, the probiotic comprises at least one of Lactobacillus rhamnosus R0011, Bifidobacterium longum R0175, and Lactobacillus helveticus R0052.

In some instances, the probiotic comprises a mixture of at least two of Lactobacillus rhamnosus R0011, Bifidobacterium longum R0175, and Lactobacillus helveticus R0052.

In some instances, the prebiotic comprises at least two of isomaltooligosaccharide, galactooligosaccharide and/or galactomannan In some, the prebiotic comprises at least about 5 grams of at least two of isomaltooligosaccharide, galactooligosaccharide and/or galactomannan

In some methods, the phytonutrient comprises at least two of apple fruit extract, grapeseed extract, and/or pine bark extract.

Some examples of the methods further include the step of identifying a subject in need of treatment. In some instances, this includes identifying a subject in need of at least one of body fat reduction, blood cholesterol reduction, blood triglyceride reduction, cortisol reduction, mood improvement and/or increases in beneficial gut bacterial species.

Novel nutritional supplements are also described. Such supplements may include a probiotic comprising at least one species of probiotic bacterium, a prebiotic comprising at mixture of at least one of isomaltooligosaccharide, galactooligosaccharide and/or galactomannan, and a phytonutrient comprising a mixture of at least one of apple fruit extract, grapeseed extract, and/or pine bark extract.

In some aspects, the probiotic comprises at least one of Lactobacillus rhamnosus, Bifidobacterium longum, and Lactobacillus helveticus. In some aspects, the Lactobacillus rhamnosus is Lactobacillus rhamnosus R0011. In some aspects, the Bifidobacterium longum is Bifidobacterium longum R0175. In some aspects, the Lactobacillus helveticus is Lactobacillus helveticus R0052.

In some aspects, the probiotic comprises a mixture of at least two of Lactobacillus rhamnosus R0011, Bifidobacterium longum R0175, and Lactobacillus helveticus R0052.

In some aspects, the prebiotic comprises at least two of isomaltooligosaccharide, galactooligosaccharide and/or galactomannan. In some aspects, the prebiotic comprises at least about 5 grams of at least two of isomaltooligosaccharide, galactooligosaccharide and/or galactomannan.

In some aspects, the phytonutrient comprises at least two of apple fruit extract, grapeseed extract, and/or pine bark extract.

In some aspects, the nutritional supplement includes a probiotic comprising at least one of Lactobacillus rhamnosus, Bifidobacterium longum, and Lactobacillus helveticus, a prebiotic comprising at mixture of at least one of isomaltooligosaccharide, galactooligosaccharide and/or galactomannan, and a phytonutrient comprising a mixture of at least one of apple fruit extract, grapeseed extract, and/or pine bark extract.

In some aspects, the Lactobacillus rhamnosus is Lactobacillus rhamnosus R0011.

In some aspects, the Bifidobacterium longum is Bifidobacterium longum R0175.

In some aspects, the Lactobacillus helveticus is Lactobacillus helveticus R0052.

In some aspects, the probiotic comprises a mixture of at least two of Lactobacillus rhamnosus R0011, Bifidobacterium longum R0175, and Lactobacillus helveticus R0052.

In some aspects, the prebiotic comprises at least two of isomaltooligosaccharide, galactooligosaccharide and/or galactomannan. In others, the prebiotic comprises at least about 5 grams of at least two of isomaltooligosaccharide, galactooligosaccharide and/or galactomannan.

In some aspects, the phytonutrient comprises at least two of apple fruit extract, grapeseed extract, and/or pine bark extract.

In some instances, the nutritional supplement includes a probiotic comprising a mixture of Lactobacillus rhamnosus R0011, Bifidobacterium longum R0175, and Lactobacillus helveticus R0052, a prebiotic comprising at mixture of isomaltooligosaccharide, galactooligosaccharide and/or galactomannan, and a phytonutrient comprising a mixture of apple fruit extract, grapeseed extract, and/or pine bark extract.

### EXAMPLE 1

Experimental Design

A study was conducted to explore the effects of supplementation using supplements and methods of supplementation according to the present disclosure. A test group of 33 subjects, including 10 men and 23 women were recruited to participate in the study. The subjects averaged 45 years old, and weighed, on average 176 pounds, with 27% body fat.

The trial subjects underwent nutritional supplementation with a nutritional supplement. The supplement included a 3 billion CFU blend of Lactobacillus rhamnosus R0011, Bifidobacterium longum R1075, and Lactobacillus helveticus R0052; a prebiotic composition including a 5-gram blend of isomaltooligosaccharide (IMO), galactooligosaccharide (GOS), and galactomannan; and phytonutrients, including a 75 mg blend of apple fruit extract, grapeseed extract, and pine bark extract.

Several physiological parameters were measured at the beginning of the study, including: body fat %, total cholesterol, LDL cholesterol, blood triglycerides, blood glucose levels, total cholesterol/HDL ratio, blood cortisol levels, butyrate kinase levels (via fecal analysis).

Each subject's gut bacteria were also characterized by DNA measurement using polymerase chain reaction (PCR) techniques. More specifically, microbiome analysis of fecal samples was carried out using the complete Biome Tracker system (Wasatch Scientific, Murray, Utah). Briefly, Fecal samples were obtained by nylon swab and placed into preservative binding buffer to lock the composition of bacteria in place. DNA was then purified by following the recommended procedure and using the provided DNA columns. Reaction mixtures were set up as recommended with the components provided (WS #1-WS #8), and ˜20 ng of DNA from each sample was added to the reaction mixtures. Samples were processed using the recommended conditions on an ABI 7500 Fast (Applied Biosystems) instrument in duplicate. Threshold values were input into the normalization/quantification template provided.

Baseline measurements of subject mood state were also taken of each subject.

Subjects then received the supplement once-daily for a period of six weeks, following which the body fat %, total cholesterol, LDL cholesterol, blood triglycerides, blood glucose levels, total cholesterol/HDL ratio, blood cortisol levels, and butyrate kinase levels were measured again. As before the study, each subject's gut bacteria were characterized. Lastly, updated measurements of subject mood state were also taken.

The results are indicated below as averages:

TABLE 1
Research Study Results

| | |
|---|---|
| Body Fat %: | −2% |
| Total Cholesterol: | −8% |
| LDL Cholesterol: | −5% |
| HDL Cholesterol: | +3% |
| Blood Triglycerides: | −23% |
| Blood Glucose: | −6% |
| Total Cholesterol/HDL Ratio: | −7% |
| Blood Cortisol: | −11% |
| Butyrate Kinase: | +89% |
| Bifidobacterium Species: | +8% |
| Lactobacillus Species: | +33% |
| Akkermansia Species: | +90% |
| S. Thermopolis Species: | +62% |
| Firmicutes/Bacteroides Ratio: | −14% |

In addition to the notable decreases in body fat %, total cholesterol and LDL cholesterol, triglycerides, blood glucose and cortisol, the markers of gastrointestinal health are positive. Specifically, butyrate kinase is used as an indicator of good gut health and metabolism. Similarly, the increases in the levels of Bifidobacterium, Lactobacillus, Akkermansia, and S. Thermopolis are indicators in positive changes in gut health. The drop in the Firmicutes/Bacteroides ratio is also recognized to correspond with a leaner metabolism.

Subjects also reported overall improvements in the measurements of mood state taken at the end of the six-week period of supplementation:

TABLE 2

| Mood State Measurements: | |
|---|---|
| Depression: | −38% |
| Confusion: | −31% |
| Fatigue: | −42% |
| Anger: | −39% |
| Tension: | −41% |
| Vigor: | +23% |
| Global Mood State/Well-Being: | +17% |

Notably, all of these reported mood state measurements moved in a positive direction, with reductions in depression, confusion, fatigue, anger, and tension, and increases in vigor and global mood state/well-being.

As used herein, including in the claims, “or” as used in a list of items (e.g., a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

The description set forth herein, in connection with the example, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

1. A method of improving microbiome balance in a subject, the method comprising:
administering to the subject an effective amount of a composition comprising:
a probiotic comprising at least one species of probiotic bacterium;
a prebiotic comprising at mixture of at least one of isomaltooligosaccharide, galactooligosaccharide and/or galactomannan; and
a phytonutrient comprising a mixture of at least one of apple fruit extract, grapeseed extract, and/or pine bark extract.

2. The method of claim 1, wherein the probiotic comprises at least one of Lactobacillus rhamnosus R0011, Bifidobacterium longum R0175, and Lactobacillus helveticus R0052.

3. The method of claim 2, wherein the probiotic comprises a mixture of at least two of Lactobacillus rhamnosus R0011, Bifidobacterium longum R0175, and Lactobacillus helveticus R0052.

4. The method of claim 1, wherein the prebiotic comprises at least two of isomaltooligosaccharide, galactooligosaccharide and/or galactomannan.

5. The method of claim 4, wherein the prebiotic comprises at least about 5 grams of at least two of isomaltooligosaccharide, galactooligosaccharide and/or galactomannan.

6. The method of claim 1, wherein the phytonutrient comprises at least two of apple fruit extract, grapeseed extract, and/or pine bark extract.

7. The method of claim 1, further comprising the step of identifying a subject in need of improvement of microbiome balance.

8. The method of claim 7, wherein identifying a subject in need of treatment includes identifying a subject in need of at least one of body fat reduction, blood cholesterol reduction, blood triglyceride reduction, cortisol reduction, mood improvement, and/or increases in beneficial gut bacterial species.

9. A method of improving microbiome balance in a subject, the method comprising:
administering to the subject an effective amount of a composition comprising:
a probiotic mixture comprising at least two of Lactobacillus rhamnosus R0011, Bifidobacterium longum R0175, and Lactobacillus helveticus R0052;
a prebiotic comprising at mixture of at least one of isomaltooligosaccharide, galactooligosaccharide and/or galactomannan; and
a phytonutrient comprising a mixture of at least one of apple fruit extract, grapeseed extract, and/or pine bark extract.

10. The method of claim 9, wherein the probiotic comprises a mixture of Lactobacillus rhamnosus R0011, Bifidobacterium longum R0175, and Lactobacillus helveticus R0052.

11. The method of claim 9, wherein the prebiotic comprises a mixture of isomaltooligosaccharide, galactooligosaccharide and galactomannan.

12. The method of claim 9, wherein the phytonutrient comprises a mixture of apple fruit extract, grapeseed extract, and/or pine bark extract.

13. The method of claim 9, further comprising the step of identifying a subject in need of improvement of microbiome balance.

14. The method of claim 13, wherein identifying a subject in need of treatment includes identifying a subject in need of at least one of body fat reduction, blood cholesterol reduction, blood triglyceride reduction, cortisol reduction, mood improvement, and/or increases in beneficial gut bacterial species.

15. (canceled)

16. A composition, comprising:
a probiotic comprising at least one species of probiotic bacterium;
a prebiotic comprising at mixture of at least one of isomaltooligosaccharide, galactooligosaccharide and/or galactomannan; and
a phytonutrient comprising a mixture of at least one of apple fruit extract, grapeseed extract, and/or pine bark extract.

17. The composition of claim 16, wherein the probiotic comprises at least one of Lactobacillus rhamnosus, Bifidobacterium longum, and Lactobacillus helveticus.

18. The method of claim 17, wherein the Lactobacillus rhamnosus is Lactobacillus rhamnosus R0011.

19. The composition of claim 17, wherein the Bifidobacterium longum is Bifidobacterium longum R0175.

20. The composition of claim 17, wherein the Lactobacillus helveticus is Lactobacillus helveticus R0052.

21. The method of claim 16, wherein the probiotic comprises a mixture of at least two of Lactobacillus rhamnosus R0011, Bifidobacterium longum R0175, and Lactobacillus helveticus R0052.

22. The method of claim 16, wherein the prebiotic comprises at least two of isomaltooligosaccharide, galactooligosaccharide and/or galactomannan.

23. The method of claim 22, wherein the prebiotic comprises at least about 5 grams of at least two of isomaltooligosaccharide, galactooligosaccharide and/or galactomannan.

24. The method of claim 16, wherein the phytonutrient comprises at least two of apple fruit extract, grapeseed extract, and/or pine bark extract.

25-33. (canceled)
